(19) United States
(12) Patent Application Publication
(10) Pub. No.: US 2002/0137106 A1
(43) Pub. Date: Sep. 26, 2002

(54) Detection of biological pathway components

(75) Inventors: Sau-Mei Leung, Clinton, MA (US); Lee Lomas, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ciphergen Biosystems, Inc., Fremont, CA

(21) Appl. No.: 10/094,006

(22) Filed: Mar. 7, 2002

Related U.S. Application Data
(60) Provisional application No. 60/274,479, filed on Mar. 9, 2001.

(51) Int. Cl. G01N 33/53; G01N 33/542; C12Q 1/68; C12M 1/34

(52) U.S. Cl. 435/7.9; 435/287.2; 435/6; 250/281

(57) ABSTRACT

The present invention provides materials and methods for simultaneously analyzing multiple components of a biological pathway (e.g., signal transduction, immunological, plasma enzyme mediated, cell cycle or developmental cycle).

### CROSS-REFERENCES TO RELATED APPLICATIONS

This patent application claims the benefit of the priority date of U.S. Provisional patent application No. 60/274,479, filed Mar. 9, 2001.

### STATEMENT AS TO RIGHTS TO INVENTIONS MADE UNDER FEDERALLY SPONSORED RESEARCH AND DEVELOPMENT

Not Applicable.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

Cause and effect in biological systems frequently occurs through biological pathways, both regulatory and metabolic, involving many components (affectors or effectors) acting in sequence. Biological pathways include, for example, signal transduction pathways, immune response pathways, plasma enzyme mediated pathways, cell cycle pathways and developmental pathways.

Signal transduction refers to a process by which an external signal is transmitted into a cell to stimulate or inhibit intracellular processes. Signal transduction is generally initiated by the interaction of various extracellular factors, such as hormones, adhesion molecules, and neurotransmitters, with effectors (e.g., receptors on the cell surface or with intracellular receptors). These extracellular signals are propagated via at least one intracellular component, causing, e.g., the intracellular domains of receptor molecules to interact with intracellular targets, such as proteins. The intracellular receptor-target interactions initiate a cascade of additional biomolecule interactions in the cell. These intracellular interactions propagate the signal throughout the cell along one or more intracellular signal transduction pathways.

Some components in the signal transduction pathways play roles in disease processes, such as cancer, allergy, arthritis, osteoporosis, and Alzheimer's disease. For example, in cancer cells, mutated versions of oncogenes and tumor suppressor genes, which are often components of signaling pathways that regulate cell growth and survival, result in uncontrollable growth of cancer cells. In another example, Alzheimer's disease involves altered regulation of various signal transduction pathway components, such as G-protein stimulated adenylate cyclase, Ins(1,4,5)P3 receptor, and protein kinase C.

Immune system pathways include those involved in both the cell-mediated and humoral effector responses, including cytotoxicity and delayed hyper sensitivity. Cytokines are involved in many of these pathways.

Plasma enzyme mediators include the kinin system, the clotting system, the fibrinolytic system and the complement system.

Current technologies, such as Western blot or two-dimensional gel analyses, do not provide the speed, sensitivity or ability to analyze multiple components of a biological pathway (e.g., signal transduction, immunological, plasma enzyme mediated, cell cycle or developmental cycle) at the same time. Therefore, there is a need to develop simpler analytical methods and materials for analyzing components of such biological pathways and for determining which component contributes to disease processes. Embodiments of the invention meet this and other goals.

### SUMMARY OF THE INVENTION

The present invention provides materials and methods for simultaneously analyzing multiple components of biological pathways, including signal transduction pathways, immune system pathways, plasma enzyme pathways, cell cycle pathways and developmental pathways using any of a number of detection methods including, but not limited to, gas phase ion spectrometry (e.g., mass spectrometry), fluorescent detection, integrated optical detection, surface plasmon resonance, ellipsometry and atomic force microscopy. Embodiments of the invention provide the speed, sensitivity and ability to analyze multiple components of a biological pathway, and are particularly useful for determining which component or components of the pathway is/are defective in a given sample.

Generally, embodiments of the invention utilize a substrate that comprises, on its surface, capture reagents that specifically bind to components of a biological pathway (e.g., signal transduction, immunological, plasma enzyme mediated, cell cycle or developmental cycle). A sample, such as cell extracts, is applied to the substrate surface and is incubated to allow binding of the components present in the sample to their corresponding capture reagents. The pathway components bound to the substrate are analyzed by the detector (e.g., a gas phase ion spectrometer). If the biological pathway is normal in the sample, then all of the components of the biological pathway are detected. However, if one component in the middle of the pathway is defective, then the defective component and other components downstream from the defective component in the pathway may not be detected. Therefore, embodiments of the invention can be used, among others, as a diagnostic tool to determine if a sample has a defect in one or more components of a biological pathway of interest.

Accordingly, in one aspect, the invention provides a substrate adapted for use with a detector, e.g., a gas phase ion spectrometer, wherein the substrate comprises at least two different capture reagents on a surface of the substrate, and wherein the capture reagents specifically bind to the different components of one or more biological pathways. In one embodiment, at least two different capture reagents are immobilized on different addressable locations on the surface of the substrate. In another embodiment, the capture reagents specifically bind to the different components of a signal transduction pathway. In another embodiment, at least two different components are sequentially activated components in a signal transduction pathway. In another embodiment, the substrate is a probe that is removably insertable into a gas phase ion spectrometer. In another embodiment, the substrate is a plurality of beads, which are placed on a probe that is removably insertable into a gas phase ion spectrometer.

In another aspect, the invention provides methods for detecting at least two components of one or more a biological pathway (e.g., signal transduction, immunological, plasma enzyme mediated, cell cycle or developmental cycle) in a sample, wherein the methods comprise: providing a substrate comprising at least two different capture reagents immobilized on a surface of the substrate, wherein the capture reagents specifically bind to the different components of the a biological pathway; contacting the sample with the substrate; and detecting the components of the biological pathway bound to their corresponding capture reagents on the substrate by gas phase ion spectrometry. In one embodiment, the gas phase ion spectrometry is mass spectrometry. In another embodiment, the mass spectrometry is laser desorption/ionization mass spectrometry. In another embodiment, the method further comprises (a) generating data on the sample with a mass spectrometer indicating intensity of signal for mass/charge ratios; (b) transforming the data into computer-readable form; and (c) operating a computer to execute an algorithm, wherein the algorithm determines closeness-of-fit between the computer-readable data and control data.

In another aspect, the invention provides kits comprising: (a) a substrate adapted for use with a detector, e.g., a gas phase ion spectrometer, the substrate comprising at least two different capture reagents immobilized on a surface of the substrate, wherein the capture reagents specifically bind to the different components of one or more a biological pathway (e.g., signal transduction, immunological, plasma enzyme mediated, cell cycle or developmental cycle); and (b) an instruction material for detecting the different components of the biological pathway by contacting a sample with the substrate and detecting the components retained by the capture reagents. In one embodiment, the kit further comprises a reference material. In another embodiment, the kit further comprises: (a) an eluant for washing the substrate, which removes unbound materials and allows retention of components of the a biological pathway (e.g., signal transduction, immunological, plasma enzyme mediated, cell cycle or developmental cycle) bound to their corresponding capture reagents; or (b) an instruction material for washing the substrate with the eluant after contacting the substrate with a sample.

### BRIEF DESCRIPTION OF THE DRAWINGS

FIG. 1 illustrates a probe adapted for use with a gas phase ion spectrometer, wherein substrate 101 is in the form of a strip, upon which a plurality of discrete spots 102, upon which capture reagents are immobilized.

FIGS. 2A-2E illustrate the Ras/Raf signal transduction pathway, capture reagents or baits that can be used to capture components of the pathway, and exemplary mass spectra.

FIGS. 3A and 3B illustrate the p53 tumor suppressor signal transduction pathway, and capture reagents or baits that can be used to capture components of the pathway.

FIGS. 4A and 4B illustrate the BRCA1 signal transduction pathway, and capture reagents of baits that can be used to capture components of the pathway.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Signal transduction” refers to a process by which the information contained in an extracellular physical or chemical signal (e.g., hormone or growth factor) is received at the cell by the activation of specific receptors and conveyed across the plasma membrane, and along an intracellular chain of various components, to stimulate the appropriate cellular response.

“Signal transduction pathway components,” “pathway components,” or “components of a signal transduction pathway” refer to intracellular or transmembrane biomolecules (of a particular apparent molecular weight) which are activated in cascade in response to an extracellular signal received by the cell.

The phrase “differentially present” refers to differences in the quantity of a signal transduction pathway component present in a test sample as compared to a control (a sample taken from a normal subject or cells).

A component of a biological pathway is differentially present between the two samples if the amount of the component in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

A “test amount” of a biological pathway component refers to an amount of the component present in a sample being tested. A test amount can be either in absolute amount (e.g., μg/ml) or in a relative amount (e.g., relative intensity of signals).

A “control amount” of a biological pathway component can be any amount or a range of amount which is to be compared against a test amount of a biological pathway component. For example, a control amount of the component can be the amount of the component in a normal cell or person, which or who is known to have an intact, functional biological pathway. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a biological pathway component for detection. A probe can comprise a single substrate or a plurality of substrates. Terms such as ProteinChip®, ProteinChip® array, or chip are also used herein to refer to specific kinds of probes.

“Substrate” or “probe substrate” refers to a solid phase onto which a capture reagent can be provided (e.g., by attachment, deposition, etc.).

“Capture reagent” refers to any material capable of specifically binding a component of a biological pathway.

“Eluant” or “washing solution” refers to a liquid that can be used to wash and remove unbound material from the substrate surface.

“Gas phase ion spectrometry” refers to a method comprising employing an ionization source to generate gas phase ions from an analyte presented on a sample presenting surface of a probe and detecting the gas phase ions with a gas phase ion spectrometer. Gas phase ion spectrometers include, for example, mass spectrometers, ion mobility spectrometers, and total ion current measuring devices.

“Laser desorption mass spectrometer” refers to a mass spectrometer which uses laser as means to desorb, volatilize, and ionize an analyte.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as nonglycoproteins.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

Methods for preparing antibodies are well-known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to Ras protein from specific species such as rat, mouse, or human can be selected to obtain only those polyclonal antibodies that are specifically immunoreactive with Ras protein and not with other proteins, except for polymorphic variants and alleles of Ras protein. This selection may be achieved by subtracting out antibodies that cross-react with Ras proteins from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Energy absorbing molecule” or “EAM” refers to a molecule that absorbs energy from an ionization source in a mass spectrometer thereby aiding desorption of analyte, such as a biological pathway component, from a probe surface. Depending on the size and nature of the analyte, the energy absorbing molecule can be optionally used. Energy absorbing molecules used in MALDI are frequently referred to as “matrix.” Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

### DETAILED DESCRIPTION

I. Introduction

Cellular signal transduction is a fundamental mechanism whereby external stimuli that regulate diverse cellular processes are relayed to the interior of cells. Cell signal transduction requires both an extracellular signaling molecule and a set of receptors in each cell to interact with one another and generate a cascade of events that result in a particular biological response (e.g., cellular response). The cellular response includes, e.g., transcription of specific genes, cell growth, cell death, cell division, cell adhesion, endocytosis, etc. Typically, each cell responds to a specific set of signals that act in various combinations to regulate various cellular responses.

There are two types of receptors in the cell that interact with extracellular signaling molecules: intracellular receptors and cell surface receptors. Extracellular signaling molecules, such as small hydrophobic molecules, diffuse across the plasma membrane of the target cell and activate intracellular receptors. Examples of these extracellular signaling molecules include, e.g., steroid, thyroid hormones, vitamin D and retinoids. These molecules directly regulate the transcription of specific genes. Other molecules, such as dissolved nitric oxide and carbon monoxide gasses diffuse across the plasma membrane and activate intracellular enzymes, typically guanylate cyclase, which produces cyclic GMP in the target cell. The increased production of cyclic GMP, in turn, produces various cellular responses. Albert et al., ed., Molecular Biology of the Cell, 3rd ed. (Garland Publishing, Inc. New York, 1994).

The majority of extracellular signaling molecules are hydrophilic and typically bind to cell surface receptors. In response to binding of extracellular signaling molecules, the cytoplasmic domain of the cell surface receptors may change conformation and transmit the signal across the membrane. Alternatively, individual receptors may aggregate and interact with other membrane proteins to generate a response. These events trigger a cascade of events in the cell, including, e.g., changes in intracellular Ca²⁺ levels, enzymatic activity and gene expression. Albert et al., supra.

Cell surface receptors can be categorized into three types, wherein each type transduces extracellular signals in a different manner. These include ion-channels, G-protein linked receptors, and enzyme-linked receptors. Ion-channel-linked receptors are receptors that bind to a ligand, and in response, open or close ligand-gated ion channels. G-protein-linked receptors indirectly activate or inactivate plasma-membrane-bound enzymes or ion channels via trimeric GTP-binding proteins (G proteins). Enzyme-linked receptors act directly as enzymes or are associated with enzymes. Albert et al., supra.

G-protein-linked receptors generate a cascade of events via small intracellular mediators, such as cAMP or Ca²⁺. In both cases, the binding of an extracellular signaling molecule to the G-protein-linked receptors changes the conformation of the cytoplasmic domain of the receptor, causing it to bind to a G protein that activates or inactivates a plasma membrane enzyme. In the cAMP pathway, the enzyme directly produces cAMP. In the Ca²⁺ pathway, the enzyme produces a soluble mediator (inositol triphosphate) that release Ca²⁺ from the endoplasmic reticulum. Both cAMP and Ca²⁺ transmit the signal by acting as allosteric effectors by binding to specific proteins in the cell, altering their conformation and thereby their activity. Albert et al., supra.

There are five known classes of enzyme-linked receptors: (1) transmembrane guanylyl cyclases, which generate cGMP directly; (2) receptor tyrosine phosphatases, which removes phosphatase from phosphotyrosine side chains of specific proteins; (3) transmembrane receptor serine/threonine kinases, which add a phosphatase group to serine and threonine side chains on target proteins; (4) receptor tyrosine kinases; and (5) tyrosinekinase-associated receptors. Albert et al., supra.

Among enzyme-linked receptors, receptor tyrosine kinases and tyrosinekinase-associated receptors are most numerous. They attach phosphate to the tyrosine residues of target proteins in cells. Tyrosine kinase receptors are activated by various extracellular signaling molecules, such as epidermal growth factor, insulin, platelet-derived growth factor and fibroblast growth factor. Binding of these factors to the kinase receptors cause the receptors to change conformation, which in turn activates the kinase activity of the receptor or its associated non-receptor tyrosine kinase. When activated, receptor tyrosine kinases usually cross-phosphorylate themselves, which then serve as binding sites for certain intracellular signaling proteins comprising SH2 domains. Through cascades of highly regulated protein phosphorylations, elaborate sets of interacting proteins relay most signals from the cell surface to the nucleus, thereby altering the cell's pattern of gene expression and its behavior. Albert et al., supra.

Sometimes one of the components in the signal transduction pathway is defective, which in turn contributes to various disease conditions, such as cancer, allergy, arthritis, etc. For example, a mutation in p53 tumor suppressor causes abnormal cell growth, which can lead to various types of cancers. In another example, a mutation in the BRCA1 gene, which is one component of a signal transduction in repairing DNA damage, can lead to breast and ovarian cancer. In some diseases, it may be uncertain as to which component of the signal transduction pathway is defective. Therefore, there is a need to develop materials and methods that allow quick and reliable analysis of multiple components of signal transduction pathways.

Embodiments of the invention provide means for simultaneously analyzing multiple components of a biological pathway (e.g., signal transduction, immunological, plasma enzyme mediated, cell cycle or developmental cycle) and for determining which component contributes to disease conditions. Generally, a sample comprising biological pathway components is applied to a substrate that comprises, on its surface, capture reagents that specifically bind to the pathway components. Then the multiple pathway components bound the substrate surface are analyzed by any of a number of methods, including gas phase ion spectrometry (e.g., mass spectrometry).

If all of the pathway components are functional and normal, they bind to their corresponding capture reagents on the substrate surface, and their signals are detected at their predicted mass values by, e.g., mass spectrometry. However, if one of the pathway components is defective, it may not bind to its capture reagent on the substrate surface, and its signal may not be detected at its predicted mass or detected with an altered signal strength (compared to control). Thus, a lack of signal from any component from the substrate surface indicates that the biological pathway has a defect at that point of the pathway.

For example, if the signal transduction pathway is a G-protein linked receptor pathway, and if there is no signal from the capture reagent that binds the G-protein linked receptor, then this lack of signal indicates that there is a defect in the G-protein linked receptor. If there is no signal from the capture reagent that binds to a plasma membrane enzyme, such as adenylate cyclase, then this lack of signal indicates that there is a defect in the plasma membrane enzyme. If there is no signal from the capture reagent that binds to a final target protein of the pathway, then this lack of signal indicates that there is a defect in the final target protein. Generally, if one point in the middle of the pathway is defective, then all of the components downstream from the defective component will also not be detected or will be detected with signal strengths that differ from a control.

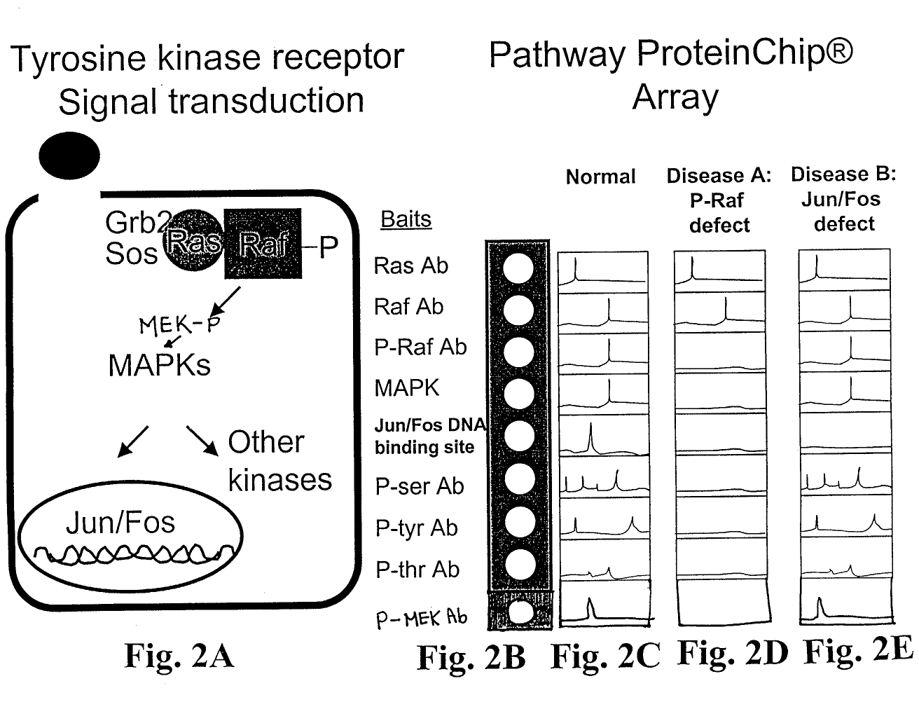

In another example, if the signal transduction pathway is a tyrosine kinase pathway and if there is no signal from the capture reagent that binds to a tyrosine kinase, then this lack of signal indicates that there is a defect in the tyrosine kinase. If there is a signal from the capture reagent that binds to a tyrosine kinase but not from the capture reagent that binds to the phosphorylated form of tyrosine kinase, then this lack of signal indicates that phosphorylation function of the kinase is defective. If there is no signal from an intracellular signaling protein with SH2 domain, then this lack of signal indicates that there is a defect in the intracellular signaling protein downstream from the tyrosine kinase. As noted above, if one point in the middle of the pathway is defective, then all of the components downstream from the defective component will also not be defected or will be detected with signal strengths that differ from a control. However, if a defect is downstream from a branch point of a signal transduction pathway (see, e.g., FIG. 2A), then there will be a lack of signal on one branch of the signal transduction pathway whereas signals will be detected on the other branch of the signal transduction pathway.

Other variations will be readily apparent to those skilled in the art and are within the scope of the present invention. The materials and methods for embodiments of the invention are described in detail below.

II. Substrates for Capturing Components of Biological Pathways

In one aspect, the invention provides substrates adapted for use with a detector, such as a gas phase ion spectrometer, wherein the substrate comprises at least two different capture reagents immobilized on the substrate surface. The capture reagents specifically bind to different components of one or more a biological pathway (e.g. signal transduction, immunological, plasma enzyme mediated, cell cycle or developmental cycle). Typically, capture reagents are located at different locations of the substrate so that one can readily distinguish which biological pathway components in a sample are bound to the substrate. Alternatively, capture reagents can be placed at the same location of the substrate as long as the pathway components have different molecular weight. The substrates and the capture reagents are described in detail below.

A. Substrates

Analytes can be captured on any of a variety of protein biochips. Among the many protein biochips described in the art are those biochips produced by Ciphergen Biosystems (Fremont, Calif.), Packard BioScience Company (Meriden Conn.), Zyomyx (Hayward, Calif.) and Phylos (Lexington, Mass.). In general, protein biochips comprise a substrate having a generally planar surface. A capture reagent is attached to the surface of the substrate. Frequently, the surface comprises a plurality of addressable locations, each of which location has the capture reagent bound there. The capture reagent can be a biological molecule, such as a polypeptide or a nucleic acid, which captures other biomolecules in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), U.S. Pat. No. 6,329,209 (Wagner et al., “Arrays of protein-capture agents and methods of use thereof,” Dec. 11, 2001), International publication WO 00/56934 (Englert et al., “Continuous porous matrix arrays,” Sep. 28, 2000).

In one embodiment the substrate is a probe that capable of being engaged by a probe interface of a mass spectrometer which positions the probe in interrogatable relationship with an ionization source The probe can be in any shape, e.g., in the form of a strip, a plate, or a dish with a series of wells. Each type of capture reagent can be immobilized at different addressable locations at the substrate surface.

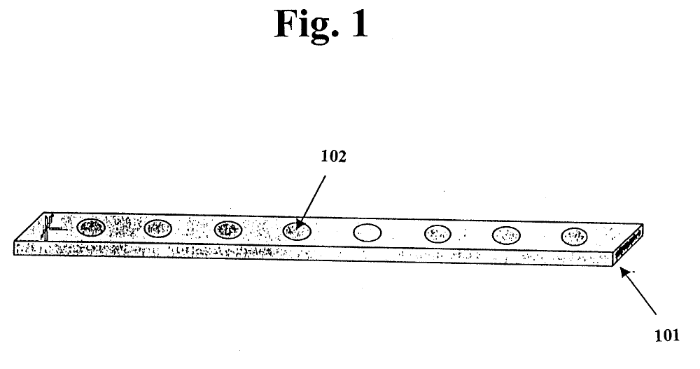

As an illustration, FIG. 1 shows probe 101 with discontinuous spots 102 on its surface, wherein each spot comprise capture reagents immobilized thereon. Typically, each spot comprises different capture reagents so that one can readily distinguish which pathway components in a sample are bound to the substrate. In some embodiments, different capture reagents can be placed at the same spot of the substrate as long as the pathway components have different molecular weight.

Each spot on the substrate is “addressable” in that during gas phase ion spectrometry, an energy source, such as a laser, is directed to, or “addresses” the spot to desorb biological pathway components bound to the capture reagents on the probe surface. The addressable locations can be arranged in any pattern on the probe surface, but are preferably in regular pattern, such as lines, orthogonal arrays, or regular curves (e.g., circles). Alternatively, capture reagents can be placed on the substrate surface in continuous patterns, rather than in discontinuous patterns as shown in FIG. 1.

Alternatively, the substrate can be a separate material that can be placed onto a probe that is removably insertable into a gas phase ion spectrometer. For example, a substrate can be a solid phase, such as a polymeric, paramagnetic, latex or glass bead, upon which are immobilized capture reagents for binding biological pathway components. The solid phase generally adheres to the probe surface, and its adherence to the probe surface can be improved by mechanical or chemical treatment of the probe surface (e.g., roughening). These solid phase materials can be placed onto a probe that is removably insertable into a gas phase ion spectrometer. The solid phase with each type of capture reagent is typically placed at different addressable locations of the probe surface. Alternatively, as noted above, different capture reagents can be placed on the same addressable locations as long as they bind to pathway components with different molecular weight.

The probe can be also shaped so that it is adapted for use with various components of a gas phase ions spectrometer, such as inlet systems and detectors. For example, the probe can be adapted for mounting in a horizontally and/or vertically translatable carriage that horizontally and/or vertically moves the probe to a successive position. This allows pathway components bound to different locations of the substrate surface to be analyzed without requiring repositioning of the probe by hand.

The probe substrate can be made of any suitable material. For example, the probe substrate material includes, but is not limited to, insulating materials (e.g., glass such as silicon oxide, plastic, ceramic), semi-conducting materials (e.g. silicon wafers), or electrically conducting materials (e.g., metals, such as nickel, brass, steel, aluminum, gold, or electrically conductive polymers), organic polymers, biopolymers, or any combinations thereof. The substrate material can also be solid or porous. Substrates suitable for use in embodiments of the invention are described in, e.g., U.S. Pat. No. 5,617,060 (Hutchens and Yip) and WO 98/59360 (Hutchens and Yip).

The probe substrate can be conditioned to bind capture reagents. In one embodiment, the surface of the probe substrate can be conditioned (e.g., chemically or mechanically such as roughening) to place capture reagents on the surface. Typically, the probe substrate comprises reactive groups that can immobilize capture reagents. For example, the probe substrate can comprise a carbonyldiimidazole group which covalently reacts with amine groups of nucleic acids or proteins, including antibodies. In another example, the probe substrate can comprise an epoxy surface which covalently reacts with amine and thiol groups of DNA and proteins. Probe substrates with these reactive surfaces are commercially available from Ciphergen Biosystems (Fremont, Calif.).

B. Capture Reagents

Intracellular biological pathway components include polypeptides, lipids, lipoproteins, carbohydrates, etc. Capture reagents can be selected from any suitable materials as long as they specifically bind to these components. For example, capture reagents are selected from polypeptides, lipids, lipoproteins, carbohydrates, nucleic acids, small organic or inorganic molecules. Typically, capture reagents are receptors, ligands, antibodies, or nucleic acids that specifically bind to components of a biological pathway.

Capture reagents are often antibodies that specifically bind to a component of a biological pathway (e.g., signal transduction, immunological, plasma enzyme mediated, cell cycle or developmental cycle). These include, e.g., monoclonal antibodies, polyclonal antibodies, antibody fragments, single chain antibodies, etc. Methods for making these antibodies are well-known in the art.

For example, monoclonal antibodies can be prepared by any technique that provides for the production of antibody molecules by continuous cell lines in culture, including the hybridoma technique originally developed by Kohler & Milstein, Nature 256:495-497 (1975), as well as the trioma technique, the human B-cell hybridoma technique (Kozbor et al., Immunology Today 4:72 (1983)), and the EBV-hybridoma technique to produce human monoclonal antibodies (Cole et al., Monoclonal Antibodies and Cancer Therapy, Alan R. Liss, Inc., pp. 77-96 (1985)).

Fragments of antibodies are also useful as binding moieties. While various antibody fragments can be obtained by the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain Fv). Single chain antibodies are also useful to construct detection moieties. Methods for producing single chain antibodies were described in, for example, U.S. Pat. No. 4,946,778. Techniques for the construction of Fab expression libraries were described by Huse et al., Science 246:1275-1281 (1989); these techniques facilitate rapid identification of monoclonal Fab fragments with the desired specificity for pathway components.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to proteins of interest. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Specific antibodies will usually bind to the target pathway component with a K_{d} of at least about 0.1 mM, more usually at least about 1 μM, preferably at least about 0.1 μM or better, and most preferably, 0.01 μM or better. Some antibodies useful for binding pathway components are also known in the art, and some are even commercially available. For example, anti-phosphothreonine antibody, anti-phosphotyrosine antibody, and anti-phospho-Raf antibody can be obtained from Cell Signaling Technology (www.cellsignal.com).

The substrate can comprise one or more different types of capture reagents immobilized on the substrate surface. Typically, at least two, three or four different capture reagents, more typically at least five to eight different capture reagents, or even hundreds or thousands of different capture reagents can be immobilized on the substrate surface. The capture reagents can be selected to bind to components of multiple biological pathways or to components of a signal transduction pathway. In some embodiments, capture reagents are selected so that they bind to two or more different components that are sequentially activated in a single signal transduction pathway as described in detail below.

Many signal transduction pathways and their components are known in the art, and the selection of capture reagents depends on analysis of which signal transduction pathway is desired. For example, capture reagents may be selected from those that selectively bind to components of the Ras/Raf signal transduction pathway, the p53 tumor suppressor signal transduction pathway, the BRCA1 signal transduction pathway, or any combinations thereof. Many other signal transduction pathways are known in the art, and are described in, e.g., Alberts et al., Molecular Biology of the Cell, 3rd ed. (Garland Publishing, Inc. New York, 1994), pages 721-785; Lodish et al., Molecular Cell Biology, 4th ed. (W.H. Freeman and Company, 2000), pages 849-906; and website www.cellsignal.com.

Typically, the capture reagents are selected so that each capture reagent binds to components of a signal transduction pathway. Even more typically, the capture reagents are selected so that at least two different capture reagents on the substrate surface bind to components that are sequential in their activation in a signal transduction pathway. Having a number of capture reagents that bind to components of a single signal transduction pathway on a substrate allows those skilled in the art to readily determine which pathway component in a sample is defective.

As an illustration, components involved in the Ras/Raf signal transduction pathway are as follows (see FIG. 2A). The signal induced by ligand binding is carried via Grb2 and Sos to Ras, leading to its activation. Activated Ras binds to the N-terminal domain of Raf, a serine/threonine kinase. Raf binds to and phosphorylates MEK, a dual-specificity protein kinase that phosphorylates both tyrosine and serine residues. MEK phosphorylates and activates MAP kinase, another serine/threonine kinase. MAP kinase phosphorylates many different proteins, including nuclear transcription factors such as Jun and Fos, which in turn mediate a cellular response.

As illustrated in FIG. 2B, if Ras/Raf signal transduction pathway of a sample is analyzed, then capture reagents that are immobilized on the substrate surface can include anti-Ras antibody, anti-Raf antibody, anti-phosphorylated Raf antibody, anti-phosphorylated MEK antibody, MAP kinase, a nucleic acid comprising Jun/Fos binding site, anti-phosphoserine antibody, anti-phosphotyrosine antibody, and anti-phosphothreonine antibody. This set of capture reagents allows one of skill in the art to determine precisely which point in the Ras/Raf signal transduction pathway may be defective in a test sample.

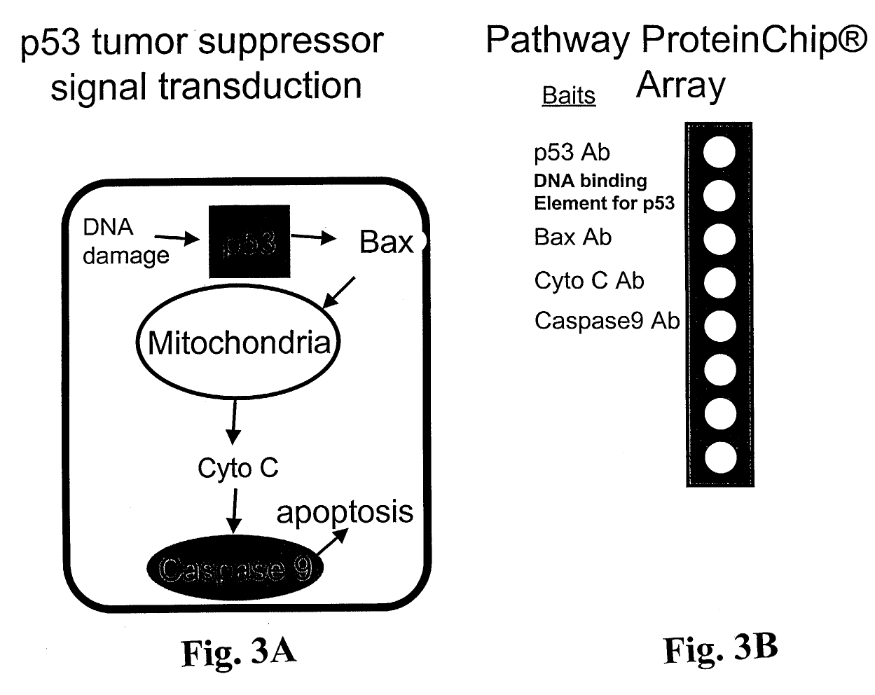

In another example, FIG. 3A illustrates the p53 tumor suppressor signal transduction pathway. P53 suppresses abnormal cell proliferation (e.g., cell damaged by DNA damage). When the p53 gene is mutated, p53 loses its ability to block abnormal cell growth. Almost 50% of human cancers including breast, colon, lung, liver, prostate, skin, contains a p53 mutation. Thus, the p53 tumor suppressor signal transduction pathway represents an important mechanism for protection against cancer. Components involved in the p53 tumor suppressor signal transduction pathway are as follows. Pro-apoptic stimuli such as DNA damage leads to the activation of p53. P53 then transcriptionally simulates Bax. Bax, in turn, causes the translocation of cytochrome C from mitochondria to cytoplasm. Then this event leads to the activation of caspase 9 which eventually leads to apoptosis or programmed cell death.

As illustrated in FIG. 3B, various capture reagents can be immobilized on the substrate surface to analyze the p53 tumor suppressor signal transduction pathway. For example, capture reagents that are immobilized on the substrate surface include anti-p53 antibody, a nucleic acid comprising p53 binding site, anti-Bax antibody, anti-cytochrome C antibody, and anti-caspase 9 antibody. These capture reagents allow one of skill in the art to determine whether a test sample has any defect in the p53 pathway.

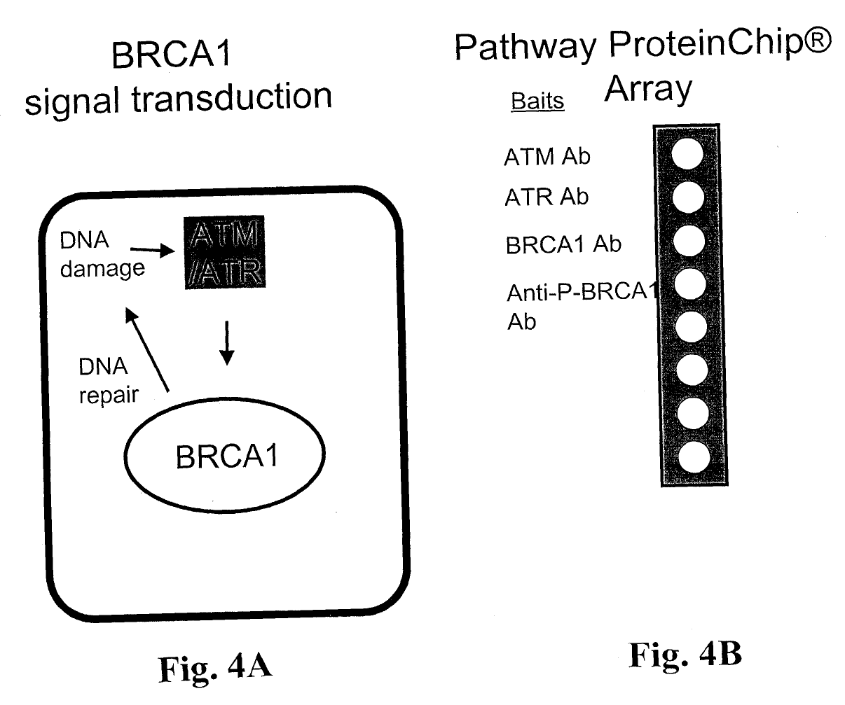

In another example, FIG. 4A illustrates the BRCA1 signal transduction pathway. BRCA1 is involved in DNA damage. Mutations in the BRCA1 gene is linked to about one-half of familial breast cancers and over 80% of families with inherited breast and ovarian cancer. As shown in FIG. 4A, components involved in the BRCA1 signal transduction pathway are as follows. DNA damage activates the ATM/ATR kinases. Then ATM/ATR phosphorylates BRCA1 which in turn repair the DNA damage.

As illustrated in FIG. 4B, various capture reagents can be immobilized on the substrate surface to analyze the BRCA1 signal transduction pathway. For example, capture reagents that are immobilized on the substrate surface can include anti-ATM antibody, anti-ATR antibody, anti-BRCA1 antibody, and anti-phosphorylated BRCA1 antibody. These capture reagents allow one of skill in the art to determine whether a test sample has any defect in the BRCA1 signal transduction pathway.

In one embodiment, the biological pathway can be the classical and/or alternative complement mediated cell lysis pathway. In another example it could be the biological pathway leading to opsonization of foreign pathogens.

In another embodiment, the biological pathway can be related to normal or non-normal cell development. In an example, this can be the dorsal-ventral patterning pathway occurring during early development. In another example this can be any transcription pathway including, but not limited to the steroid receptor superfamily of transcription factors. The can include the retinoic acid receptor (RAR), the dimerization partner of RAR (RXR) and the orphan receptor family (ROR). In another example this pathway can lead to programmed cell death (apoptosis) via both intrinsic and extrinsic mechanisms. In extrinsic cell death, this pathway involves a cascade of events initiating at the activation of a ‘death domain’ via membrane receptor-ligand binding. Signal is transduced across the cellular membrane and initiates a number of cause-and-effect associations in a defined sequential fashion that ultimately leads to cell death.

In another embodiment, the biological pathway can be related to normal or non-normal cell cycling. This includes, but is not limited to, pathways that involve the molecular pathways determining if and when cells progress through the G1-, S-, G2- and M-phases of cell growth and differentiation.

In another embodiment, the biological pathway can be related to normal or non-normal physiological maintenance. An example can include the biological pathway leading to normal blood clotting. This can be exemplified by the extreme examples of blood clotting due to injury and a lack of appropriate blood clotting as in the case of hemophilia. Another example can include the positive and negative feedback mechanisms for the normal control of blood pressure and the development/monitoring of hypertension. In another example, the biological pathway can involve regulation of insulin, glucagon, gastrin and somatostatin by the pancreas to regulate aspects of metabolism. Diseases caused by abnormal glucose maintenance include, but are not limited to diabetes. In another example, the biological pathway can involve the peptide-hormonal regulatory pathways of the pituitary gland and the hormones that are produced by the pituitary gland (eg ACTH, ADH, TSH, GH, LH, FSH, MSH, oxytocin, PRL and vasopressin). These pathways can include those associated with disease stages (e.g. abnormal titers of ACTH and TSH can lead to Cushing's syndrome and hyperthyroidism respectively).

The above examples are merely illustrative, and one of skill in the art will readily recognize that other capture reagents can be immobilized on the substrate surface to analyze other signal transduction pathways.

III. Methods for Detecting Components of a Biological Pathway

Methods detecting analytes captured on a solid substrate can generally be divided into photometric methods of detection and non-photometric methods of detection.

Photometric methods of detection include, without limitation, those methods that detect or measure absorbance, fluorescence, refractive index, polarization or light scattering. Methods involving absorbance include measuring light absorbance of an analyte directly (increased absorbance compared to background) or indirectly (measuring decreased absorbance compared to background). Measurement of ultraviolet, visible and infrared light all are known. Methods involving fluorescence also include direct and indirect fluorescent measurement. Methods involving fluorescence include, for example, fluorescent tagging in immunological methods such as ELISA or sandwich assay. Methods involving measuring refractive index include, for example, surface plasmon resonance (“SPR”), grating coupled methods (e.g., sensors uniform grating couplers (wavelength-interrogated optical sensors (“WIOS”) and chirped grating couplers), resonant mirror and interferometric techniques. Methods involving measuring polarization include, for example, ellipsometry. Light scattering methods (nephelometry) also are used.

Non-photometric method of detection include, without limitation, gas phase ion spectrometry, atomic force microscopy and multipolar coupled resonance spectroscopy. Gas phase ion spectrometers include mass spectrometers, ion mobility spectrometers and total ion current measuring devices.

Mass spectrometers measure a parameter which can be translated into mass-to-charge ratios of ions. Generally ions of interest bear a single charge, and mass-to-charge ratios are often simply referred to as mass. Mass spectrometers include an inlet system, an ionization source, an ion optic assembly, a mass analyzer, and a detector. Several different ionization sources have been used for desorbing and ionizing analytes from the surface of a probe or biochip in a mass spectrometer. Such methodologies include laser desorption/ionization (MALDI, SELDI), fast atom bombardment, plasma desorption and secondary ion mass spectrometers. In such mass spectrometers the inlet system comprises a probe interface capable of engaging the probe and positioning it in interrogatable relationship with the ionization source and concurrently in communication with the mass spectrometer, e.g., the ion optic assembly, the mass analyzer and the detector.

Solid substrates for use in bioassays that have a generally planar surface for the capture or modification of analytes and adapted for facile use as probes with detection instruments are generally referred to as biochips. Protein biochips are biochips adapted for use in the detection of peptides or proteins or analytes captured by proteins.

In another aspect, the invention provides methods for detecting components of a biological pathway, e.g., a signal transduction pathway, wherein the methods comprise: providing a substrate comprising at least two different capture reagents immobilized on a surface of the substrate, wherein the capture reagents specifically binds to the different components of the biological pathway, contacting the sample with the substrate, and detecting the components of the biological pathway bound to their corresponding capture reagents on the substrate by gas phase ion spectrometry. In some embodiments, data generated by gas phase ion spectrometry from a test sample can be compared to a control to determine if there is any defect in the biological pathway in the test sample. The sample preparation methods and gas phase ion spectrometry analysis are described in detail below.

A. Sample Preparation and Contacting the Sample to the Substrate

The sample used in this invention can be derived from any biological material sources. These include, e.g., body fluids such as blood, serum, saliva, or extracts from biological samples, such as cell lysates. Preferably, the sample is in liquid form.

The sample is contacted with a substrate comprising an capture reagent in any suitable manner, e.g., bathing, soaking, dipping, spraying, washing over, or pipetting, etc. Generally, a volume of sample containing from a few attomoles to 100 picomoles of signal transduction pathway component in about 1 μl to 500 μl is sufficient for binding to the capture reagent. The sample can contact the probe substrate comprising capture reagents for a period of time sufficient to allow the pathway components to bind to the capture reagents. Typically, the sample and the substrate comprising the capture reagents are contacted for a period of between about 30 seconds and about 12 hours, and preferably, between about 30 seconds and about 15 minutes. Typically, the sample is contacted to the probe substrate under ambient temperature and pressure conditions. For some samples, however, modified temperature (typically 4° C. through 37° C.) and pressure conditions can be desirable, which conditions are determinable by those skilled in the art.

After the substrate contacts the sample or sample solution, it is preferred that unbound materials on the substrate surface are washed out so that only the bound materials remain on the substrate surface. Washing a substrate surface can be accomplished by, e.g., bathing, soaking, dipping, rinsing, spraying, or washing the substrate surface with an eluant. A microfluidics process is preferably used when an eluant is introduced to small spots of capture reagents on the probe. Typically, the eluant can be at a temperature of between 0° C. and 100° C., preferably between 4° C. and 37° C. In some embodiments, washing unbound materials from the probe surface may not be necessary if pathway components bound on the probe surface can be resolved by gas phase ion spectrometry without a wash.

Any suitable eluants (e.g., organic or aqueous) that preserve the biologically relevant interaction can be used to wash the substrate surface. Preferably, an aqueous solution is used. Exemplary aqueous solutions include, e.g., a HEPES buffer, a Tris buffer, or a phosphate buffered saline, etc. To increase the wash stringency of the buffers, additives can be incorporated into the buffers. These include, but are limited to, ionic interaction modifier (both ionic strength and pH), water structure modifier, hydrophobic interaction modifier, chaotropic reagents, affinity interaction displacers. Specific examples of these additives can be found in, e.g., PCT publication WO98/59360 (Hutchens and Yip). The selection of a particular eluant or eluant additives is dependent on experimental conditions (e.g., types of capture reagents used or biological pathway (e.g., signal transduction, immunological, plasma enzyme mediated, cell cycle or developmental cycle) components to be detected), and can be determined by those of skill in the art.

Prior to desorption and ionization of biological pathway (e.g., signal transduction, immunological, plasma enzyme mediated, cell cycle or developmental cycle) components from the probe surface, an energy absorbing molecule (“EAM”) or a matrix material is typically applied to biological pathway components on the substrate surface. The energy absorbing molecules can assist absorption of energy from an energy source from a gas phase ion spectrometer, and can assist desorption of biological pathway components from the probe surface. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in the art. See, e.g., U.S. Pat. No. 5,719,060 (Hutchens & Yip) for additional description of energy absorbing molecules.

The energy absorbing molecule and the sample containing biological pathway components can be contacted in any suitable manner. For example, an energy absorbing molecule is mixed with the sample, and the mixture is placed on the substrate surface. In another example, an energy absorbing molecule can be placed on the substrate surface prior to contacting the substrate surface with the sample. In another example, the sample can be placed on the substrate surface prior to contacting the substrate surface with an energy absorbing molecule. Then the biological pathway components bound to the capture reagents on the substrate surface are desorbed, ionized and detected as described in detail below.

B. Desorption/Ionization and Detection

Biological pathway (e.g., signal transduction, immunological, plasma enzyme mediated, cell cycle or developmental cycle) components bound on the substrate surface can be detected by any of the ways described herein. In one embodiment, the method involves desorption and ionization of the analyte and detection the desorbed and ionized analytes. Any suitable gas phase ion spectrometers can be used as long as it is coupled with a desorption/ionization source. Preferably, a gas phase ion spectrometers is selected so that it allows quantitation of biological pathway components in the sample.

In one embodiment, a gas phase ion spectrometer is a mass spectrometer. In a typical mass spectrometer, a probe comprising biological pathway components is introduced into an inlet system of the mass spectrometer. The inlet system in this case is a probe interface connected to the mass spectrometer that engages the probes and positions it so that surface features where analyte is bound can be addressed by the ionization source, e.g., a laser, and the resulting ions are in communication with the mass spectrometer. The biological pathway components are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of signal transduction pathway components will typically involve detection of signal intensity. This, in turn, can reflect the quantity and characteristics of biological pathway components bound to the substrate. Any of the parts of a mass spectrometer (e.g., a desorption source, a mass analyzer, a detector, etc.) can be combined with other suitable parts described herein or others known in the art in embodiments of the invention.

Preferably, a laser desorption time-of-flight mass spectrometer is used in embodiments of the invention. In laser desorption mass spectrometry, a probe substrate comprising biological pathway (e.g., signal transduction, immunological, plasma enzyme mediated, cell cycle or developmental cycle) components is introduced into an inlet system. The pathway components are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of pathway components of specific mass to charge ratio.

In another embodiment, an ion mobility spectrometer can be used to detect signal transduction pathway components. The principle of ion mobility spectrometry is based on different mobility of ions. Specifically, ions of a sample produced by ionization move at different rates, due to their difference in, e.g. mass, charge, or shape, through a tube under the influence of an electric field. The ions (typically in the form of a current) are registered at the detector which can then be used to identify pathway components in a sample. One advantage of ion mobility spectrometry is that it can operate at atmospheric pressure.

In yet another embodiment, a total ion current measuring device can be used to detect and characterize signal transduction pathway components. This device can be used when the substrate has only a single type of protein. When a pathway component is present on the substrate, the total current generated from the ionized pathway component reflects the quantity and other characteristics of the component. The total ion current produced by the pathway component can then be compared to a control (e.g., a total ion current of a known compound). The quantity or other characteristics of the pathway component can then be determined.

C. Analysis of Data

Data generated by desorption and detection of biological pathway (e.g., signal transduction, immunological, plasma enzyme mediated, cell cycle or developmental cycle) components can be analyzed using any suitable means. In one embodiment, data is analyzed with the use of a programmable digital computer. The computer program generally contains a readable medium that stores codes. Certain code can be devoted to memory that includes the location of each feature on a probe, the identity of the capture reagents at that feature and the elution conditions used to wash the substrate surface. The computer also contains code that receives as input, data on the strength of the signal at various molecular masses received from a particular addressable location on the probe. This data can indicate the number of pathway components detected, including the strength of the signal generated by each component.

Data analysis can include the steps of determining signal strength (e.g., height of peaks) of biological pathway (e.g., signal transduction, immunological, plasma enzyme mediated, cell cycle or developmental cycle) components detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each pathway component or other biomolecules can be displayed in the form of relative intensities in the scale desired (e.g., 100). Alternatively, a standard may be admitted with the sample so that a peak from the standard can be used as a reference to calculate relative intensities of the signals observed for each biological pathway component detected.

The computer can transform the resulting data into various formats for displaying. In one format, referred to as “spectrum view or retentate map,” a standard spectral view can be displayed, wherein the view depicts the quantity of biological pathway (e.g., signal transduction, immunological, plasma enzyme mediated, cell cycle or developmental cycle) component reaching the detector at each particular molecular weight. In another format, referred to as “peak map,” only the peak height and mass information are retained from the spectrum view, yielding a cleaner image and enabling pathway components with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting pathway components which are up- or down-regulated compared to control. Profiles (spectra) from any two samples may be compared visually. In yet another format, Spotfire Scatter Plot can be used, wherein pathway components that are detected are plotted as a dot in a plot, wherein one axis of the plot represents the apparent molecular weight of the biological pathway (e.g., signal transduction, immunological, plasma enzyme mediated, cell cycle or developmental cycle) components detected and another axis represents the signal intensity of components detected. For each sample, pathway components that are detected and the amount of pathway components present in the sample can be saved in a computer readable medium. This data can then be compared to a control (e.g., a profile or quantity of pathway components detected in control, e.g., from healthy subjects).

D. Comparing Test Sample Data to Control

Data generated by desorption and detection of biological pathway (e.g., signal transduction, immunological, plasma enzyme mediated, cell cycle or developmental cycle) components in a test sample can be compared to a control data to determine if the biological pathway in the test sample is normal. A control data refers to data obtained from comparable samples from a normal cell or person, which or who is known to have no defects in the biological pathway. For each component of the biological pathway being analyzed, a control amount of each component from a normal sample is determined. Preferably, the control amount of each biological pathway component is determined based upon a significant number of samples taken from normal cells or persons so that it reflects variations of the amount of these components seen in the normal cell or population.

If the test amount of a particular biological pathway component is significantly increased or decreased compared to the control amount of the component, then this is a positive indication that the test sample has a defect in the biological pathway. For example, if the test amount of a biological pathway component is increased or decreased by at least 1.5 fold, 2 fold, 5 fold or 10 fold compared to the control amount, then this is an indication that the test sample has a defect in the biological pathway. In some circumstances, if defect is severe, certain components of the pathway may be undetectable.

As an illustration, FIG. 2B shows eight different capture reagents immobilized on a substrate, wherein the capture reagents bind to different components in the Ras/Raf signal transduction pathway. FIG. 2C shows exemplary mass spectra of a control sample. As shown in the mass spectra, all of the components of the Ras/Raf signal transduction pathway (i.e., Ras, Raf, phosphorylated Raf, phosphorylated MEK, MAP kinase, Jun/Fos protein, phosphoserine proteins, phosphotyrosine proteins, phosphothreonine proteins) are detected at their predicted molecular weight. The peak represents the amount of each component found in the control sample.

FIG. 2D shows exemplary mass spectra of diseased sample A. As shown in FIG. 2D, Ras and Raf are detected in the test sample. However, none of the components downstream from Raf are detected. This indicates that Raf is mutated and cannot be phosphorylated. Thus, if one component in the middle of the pathway is defective, then in some circumstances, all of the downstream components will not be detected or will be detected at an amount that differs significantly from the control amount.

FIG. 2E shows exemplary mass spectra of diseased sample B. As shown in FIG. 2E, all of the components of the signal transduction pathway are detected except Jun/Fos. This indicates that there is a defect in Jun/Fos. Since Jun/Fos are activated after a branch point of the Ras/Raf signal transduction pathway (i.e., a branch point at MAPK), other branch is not affected by the defect in Jun/Fos and other kinases can still phosphorylate other proteins. This is indicated by their binding to anti-phosphoserine antibodies, antiphosphotyrosine antibodies, and anti-phosphothreonine antibodies.

Data generated by the detector, e.g., the mass spectrometer, can then be analyzed by a computer software. The software can comprise code that converts signal from the detector into computer readable form. The software also can include code that applies an algorithm to the analysis of the signal to determine whether the signal represents a “peak” in the signal corresponding to a signal transduction pathway component. The software also can include code that executes an algorithm that compares signal from a test sample to a typical signal characteristic of “normal” and determines the closeness of fit between the two signals. The software also can include code indicating whether the test sample has a normal profile of the signal transduction pathway or if it has a defect, and which component(s) in the pathway are defective.

IV. Kits

In yet another aspect, the invention provides kits comprising a substrate adapted for use with a detection system such as a gas phase ion spectrometer fluorescence detector, integrated optical detection system, ellipsometry detection system or atomic force microscopy detection system and an instruction material for using the kit to detect components of a biological pathway (e.g., signal transduction, immunological, plasma enzyme mediated, cell cycle or developmental cycle). The kits of the invention have many applications. For example, the kits can be used to determine if a test sample has normal or defective biological pathway. In another example, the kits can be used to identify compounds that modulate the expression of one or more components of a biological pathway, e.g., a signal transduction pathway in in vitro or in vivo. In another example, kits can predict the outcome of a biological pathway based on the presence or absence of one component, or altered activity of one component.

In one embodiment, a kit comprises: (a) a substrate adapted for use with a gas phase ion spectrometer, the substrate comprising at least two different capture reagents immobilized on a surface of the substrate, wherein the capture reagents specifically bind to the different components of one of more a biological pathway (e.g., signal transduction, immunological, plasma enzyme mediated, cell cycle or developmental cycle); and (b) an instruction material for detecting the different components of the a biological pathway (e.g., signal transduction, immunological, plasma enzyme mediated, cell cycle or developmental cycle) by contacting a sample with the substrate and detecting the components retained by the capture reagents. An instruction material can be in the form of a label on the package or a separate insert material.

In some embodiments, the kit may comprise an eluant (as an alternative or in combination with an instruction material) for washing the substrate, which eluant allows retention of components of the biological pathway on their corresponding capture reagents when washed with eluant. Alternatively or additionally, the kit may further comprise an instruction material for washing the substrate with the eluant after contacting the substrate with a sample. Such kits can be prepared from the materials described above, and the previous discussion of these materials (e.g., probe substrates, capture reagents, washing solutions, etc.) is fully applicable to this section and will not be repeated.

Optionally, the kit may further comprise a standard or control information so that the test sample can be compared with the control information standard to determine if the components of a biological pathway (e.g., signal transduction, immunological, plasma enzyme mediated, cell cycle or developmental cycle) detected in a test sample are normal or defective. For example, a standard can be bovine insulin, bovine serum albumin, etc.

The present invention provides novel materials and methods for detecting components of a biological pathway (e.g., signal transduction, immunological, plasma enzyme mediated, cell cycle or developmental cycle) using any of a number of detection systems including, but not limited to mass spectrometry. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

What is claimed is:

1. A substrate adapted for use with a mass spectrometer, the substrate comprising:
at least two different capture reagents on a surface of the substrate, wherein the capture reagents specifically bind to different components of a biological pathway.

2. The substrate of claim 1, wherein the substrate is a plurality of beads that are placed on a probe that is removably insertable into a mass spectrometer.

3. The substrate of claim 1, wherein the at least two different capture reagents are immobilized on different addressable locations on the surface of the substrate.

4. The substrate of claim 1, wherein at least three different capture reagents are immobilized on the surface of the substrate.

5. The substrate of claim 1, wherein at least four different capture reagents are immobilized on the surface of the substrate.

6. The substrate of claim 1, wherein the capture reagents specifically bind to the different components of the biological pathway.

7. The substrate of claim 6, wherein the at least two different components are sequentially activated components in the biological pathway.

8. The substrate of claim 6 or 7, wherein the biological pathway is a signal transduction pathway.

9. The substrate of claim 6 or 7, wherein the biological pathway is an immune response pathway, a plasma enzyme mediated pathway, a cell cycle pathway or a developmental pathway.

10. The substrate of claim 8, wherein the signal transduction pathway is the Ras-Raf signal transduction pathway.

11. The substrate of claim 10, wherein the capture reagents are selected from the group consisting of anti-Ras antibody, anti-Raf antibody, anti-phosphorylated Raf antibody, anti-phosphorylated MEK antibody, MAP kinase, a nucleic acid comprising Jun/Fos binding site, anti-phosphoserine antibody, anti-phosphotyrosine antibody, and anti-phophothreonine antibody.

12. The substrate of claim 8, wherein the signal transduction pathway is the p53 tumor suppressor signal transduction pathway.

13. The substrate of claim 12, wherein the capture reagents are selected from the group consisting of anti-p53 antibody, a nucleic acid comprising p53 binding site, anti-Bax antibody, anti-cytochrome C antibody, and anti-caspase 9 antibody.

14. The substrate of claim 8, wherein the signal transduction pathway is the BRCA1 signal transduction pathway.

15. The substrate of claim 14, wherein the capture reagents are selected from the group consisting of anti-ATM antibody, anti-ATR antibody, anti-BRCA1 antibody, and anti-phosphorylated BRCA1 antibody.

16. The substrate of claim 1, wherein at least one capture reagent is an antibody.

17. The substrate of claim 9 wherein the plasma enzyme-mediated pathway is selected from a kinin pathway, a clotting pathway, a fibrinolytic pathway and a complement pathway.

18. A method for detecting at least two components of a biological pathway in a sample, the method comprising:
providing a substrate comprising at least two different capture reagents immobilized on a surface of the substrate, wherein the capture reagents specifically bind to the different components of the biological pathway;
contacting the sample with the substrate; and
detecting the components of the biological pathway bound to their corresponding capture reagents on the substrate.

19. The method of claim 18, wherein the components are detected by mass spectrometry.

20. The method of claim 18, wherein the at least two different capture reagents are immobilized on different addressable locations on the surface of the substrate.

21. The method of claim 19, wherein the mass spectrometry is laser desorption/ionization mass spectrometry.

22. The method of claim 18, wherein the substrate is a probe that is removably insertable into a mass spectrometer.

23. The method of claim 18, wherein the substrate is a plurality of beads, which are placed on a probe that is removably insertable into a mass spectrometer either before or after contacting the sample with the substrate.

24. The method of claim 18, wherein at least three different capture reagents are immobilized on the surface of the substrate.

25. The method of claim 18, wherein the capture reagents specifically bind to the different components in a biological pathway.

26. The method of claim 25, wherein the at least two different components are sequentially activated components in the biological pathway.

27. The method of claim 18, wherein at least one capture reagent is an antibody.

28. The method of claim 18, wherein the sample is a cell lysate.

29. The method of claim 19, the method further comprising comparing data generated by mass spectrometry to a control.

30. The method of claim 18, the method further comprising:
(a) generating data on the sample with a mass spectrometer indicating intensity of signal for mass/charge ratios;
(b) transforming the data into computer-readable form; and
(c) operating a computer to execute an algorithm, wherein the algorithm determines closeness-of-fit between the computer-readable data and control data.

31. The method of claim 18 wherein the components are detected by absorbance detection, fluorescence detection, surface plasmon resonance, refractive index detection, ellipsometry or atomic force microscopy.

32. A kit comprising:
(a) a substrate adapted for use with a mass spectrometer, the substrate comprising at least two different capture reagents immobilized on a surface of the substrate, wherein the capture reagents specifically bind to the different components of a signal transduction pathway; and
(b) an instruction material for detecting the different components of the biological pathway by contacting a sample with the substrate and detecting the components retained by the capture reagents.

33. The kit of claim 32, wherein the at least two different capture reagents are immobilized on different addressable location on the surface of the substrate.

34. The kit of claim 32, wherein at least three different capture reagents are immobilized on the surface of the substrate.

35. The kit of claim 32, wherein the capture reagents specifically bind to the different components in the biological pathway.

36. The kit of claim 35, wherein the at least two different components are sequentially activated components in the biological pathway.

37. The kit of claim 32, wherein the kit further comprises a reference material.

38. The kit of claim 32, wherein the kit further comprises:
(a) an eluant for washing the substrate, which removes unbound materials and allows retention of components of the signal transduction pathways bound to their corresponding capture reagents; or
(b) an instruction material for washing the substrate with the eluant after contacting the substrate with a sample.
